(19) United States
(12) Patent Application Publication
(10) Pub. No.: US 2004/0096933 A1
(43) Pub. Date: May 20, 2004

(54) Method of preparation of oligosaccharides

(76) Inventors: Yunping Huang, Urbana, IL (US); Tomonori Konse, Hiratsuka-shi (JP); Yehia S. Mechref, Bloomington, IN (US); Milos V. Novotny, Bloomington, IN (US)

Correspondence Address: BARNES & THORNBURG, 11 SOUTH MERIDIAN, INDIANAPOLIS, IN, 46204

(21) Appl. No.: 10/664,462

(22) Filed: Sep. 19, 2003

Related U.S. Application Data
(60) Provisional application No. 60/426,861, filed on Nov. 15, 2002.

(51) Int. Cl. C12P 21/06; C12P 19/04; C08B 37/00

(52) U.S. Cl. 435/68.1; 435/101; 536/53

(57) ABSTRACT

The invention provides a method of cleaving an O-linked oligosaccharide from a glycoprotein. The method comprises the steps of contacting a composition comprising a glycoprotein, wherein the glycoprotein comprises O-linked oligosaccharides, with a solution comprising a borane-ammonia complex to form a mixture comprising the glycoprotein and the borane-ammonia complex, incubating the mixture for a period of time sufficient to cleave the linked oligosaccharides from the glycoprotein, and forming a mixture comprising oligosaccharide alditol products and deglycosylated protein by-products.

This application claims priority under 35 U.S.C. § 19(e) to U.S. Provisional Application Serial No. 60/426,861, filed on Nov. 15, 2002, the disclosure of which is hereby incorporated by reference herein.

### GOVERNMENT RIGHTS

Research relating to this invention was supported in part by the U.S. Government under Grant No. GM24349 awarded from the National Institute of Health. The U.S. Government may have certain rights in this invention.

### FIELD OF THE INVENTION

The present invention is related to a method of cleaving oligosaccharides from glycoproteins. More particularly, the invention is related to a method for cleaving oligosaccharides from glycoproteins using a borane-ammonia complex and forming oligosaccharide alditols.

### BACKGROUND AND SUMMARY OF THE INVENTION

Structural analysis of glycoproteins has been among the most important tasks of biomedical research for a number of years. In particular, structural analysis of the oligosaccharide component (i.e., glycan component) of glycoproteins is important to biomedical research because oligosaccharides are major recognition molecules. A large number of structural variations of oligosaccharides exist because oligosaccharides are highly branched and their monosaccharide building blocks are connected by a variety of linkages. Thus, glycoproteins and proteoglycans are extremely versatile molecules in various biological processes because of their oligosaccharide components. For example, glycoproteins are essential to processes as diverse as fertilization, the immune response, blood clotting and inflammation, cell-cell recognition, infectious diseases, cellular development and differentiation, and the like.

Recently, the scope of structural analysis of the oligosaccharide components of glycoproteins has been significantly enhanced through the availability of new mass-spectrometric (MS) methods and instrumentation. Due to its high30 throughput nature and high sensitivity, the development of matrix-assisted laser desorption/ionization mass spectrometry (MALDI/MS) is a particularly significant advancement in investigating glycoprotein structure.

Sample preparation procedures are crucial to the success of MS-based analysis of the oligosaccharide component of glycoproteins (e.g., mapping and sequencing of oligosaccharides). High-sensitivity MS measurements can be seriously impaired by a low concentration of oligosaccharides in the sample used for MS and/or by limitations in oligosaccharide cleavage conditions that lead to sample contamination which interferes with MS measurements. For the class of asparagine-linked (i.e., N-linked) oligosaccharides, the feasibility of on-plate enzymatic cleavage with N-glycanase and subsequent sequencing with various exoglycosidases is possible. Using this approach, only low-microgram amounts of N-linked oligosaccharides are needed for a complete structural analysis.

With the increasing realization that threonine- and serine-linked oligosaccharides (i.e., the O-linked oligosaccharides) are implicated in important regulatory processes and in disease conditions, high-sensitivity measurements to analyze the structure of O-linked oligosaccharides will also be required for this class of oligosaccharides. Due to the limited availability and specificity of O-glycanases, certain chemical cleavages have typically been used to release O-glycans from their respective glycoproteins (e.g., β-elimination).

Alkaline β-elimination has been used during recent years in cases where sufficient quantities (e.g., milligram to gram amounts) of glycoproteins are available. Using this approach, the base-labile O-glycosidic linkages between GalNAc (i.e., N-acetylgalactosamine) and serine/threonine residues of the protein backbone are cleaved under mild alkaline conditions. In a typical alkaline β-elimination procedure, the glycoprotein samples are treated with dilute sodium hydroxide (0.05-0.1 M) solution containing excess sodium borohydride (0.8-1.0 M). The role of the sodium hydroxide is to cleave the oligosaccharides from the protein portion of the glycoproteins and the role of sodium borohydride is to convert the released oligosaccharides to the base-stable alditols, preventing a further degradation of the glycans by “peeling reactions” (i.e. sequential degradation of oligosaccharides) under alkaline conditions. Unfortunately, for small amounts of glycoprotein samples, the high residual salts make the classical β-elimination procedure incompatible with MS analysis. The typical steps for removing the salts can lead to undesirable losses of oligosaccharides. An alternative to the alkaline β-elimination protocol described above is the use of aqueous ammonia for the β-elimination of O-linked oligosaccharides from glycoproteins.

The present invention is directed to a new method for β-elimination of oligosaccharides from glycoproteins employing an ammonia-borane complex in which oligosaccharides are cleaved from glycoproteins by β-elimination, and the borane-ammonia complex acts as a reducing agent converting the cleaved oligosaccharides to their corresponding alditols (see FIG. 1). These reactions can be used, for example, to cleave O-linked oligosaccharides from low-microgram to sub-microgram amounts of glycoproteins. Both the residual ammonia and borane-ammonia complex can be easily removed through evaporation, reducing significantly the sample handling problems after the cleavage reaction. Thus, the procedure results in minimum sample purification and sample loss, and, consequently, an enhancement of the capacity for structural analysis of oligosaccharides by MS.

In one embodiment, the invention provides a method of cleaving O-linked oligosaccharides from glycoproteins. The method comprises the steps of contacting a composition comprising a glycoprotein, wherein the glycoprotein comprises O-linked oligosaccharides, with a solution comprising a borane-ammonia complex to form a mixture comprising the glycoprotein and the borane-ammonia complex, incubating the mixture for a period of time sufficient to cleave linked oligosaccharides from the glycoprotein, and forming a mixture comprising oligosaccharide alditol products and deglycosylated protein by-products.

In another embodiment, the method further comprises the step of separating at least one cleaved oligosaccharide product from the other oligosaccharide products.

In yet another embodiment, the method further comprises the step of separating at least one cleaved oligosaccharide product from at least one protein byproduct.

In still another embodiment, the method further comprises the step of analyzing the structure of the oligosaccharide products.

### BRIEF DESCRIPTION OF THE DRAWINGS

FIG. 1 shows a scheme for cleavage of O-linked or N-linked oligosaccharides from glycoproteins.

FIG. 2 shows a negative-ion matrix-assisted laser desorption/ionization time-of-flight (MALDI/TOF) mass spectrum of O-glycans cleaved from calf serum fetuin by β-elimination.

FIG. 3 shows a MALDI/TOF mass spectrum of O-glycans cleaved from bovine submaxillary mucin by 13-elimination. FIG. 3a shows the negative-ion mode and FIG. 3b shows the positive-ion mode.

FIG. 4 shows a MALDI/TOF mass spectrum of O-glycans cleaved from human milk bile salt-stimulated lipase by β-elimination. FIG. 4a shows the negative-ion mode and FIG. 4b shows the positive-ion mode.

### DETAILED DESCRIPTION OF THE INVENTION

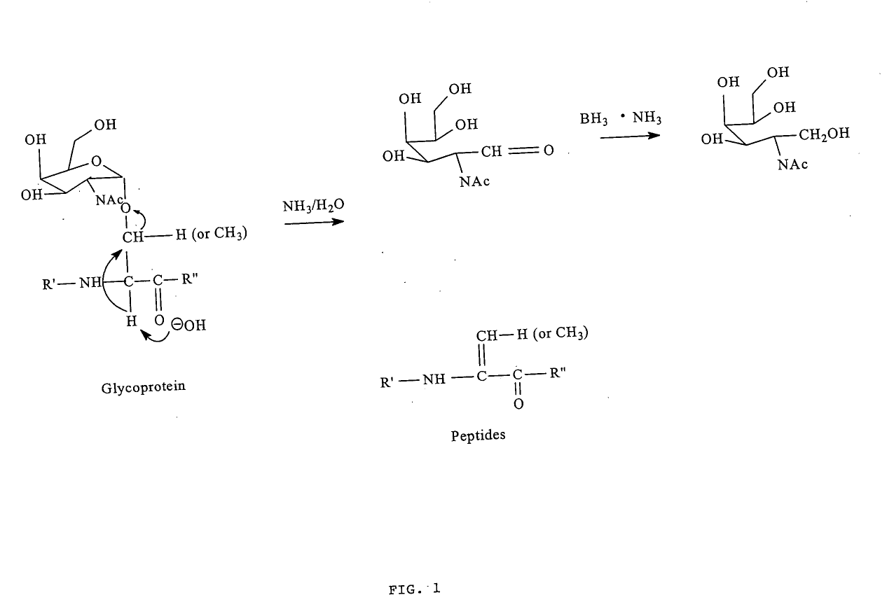

The present invention is directed to the use of a new method for β-elimination of oligosaccharides from glycoproteins employing an ammonia-borane complex in which the oligosaccharides are cleaved from glycoproteins by β-elimination, and the borane-ammonia complex acts as a reducing agent (see FIG. 1) to converting the cleaved oligosaccharides to their corresponding alditols. These reactions can be used, for example, to cleave O-linked oligosaccharides from low-microgram to sub-microgram amounts of glycoproteins.

While the availability of N-glycanases has secured “clean” glycan samples for enzymatic sequencing of N-linked oligosaccharides using small amounts of glycoproteins as the starting material, a comparable situation has not existed for the biologically significant and ubiquitous O-linked oligosaccharides. The necessity of using chemically-based rather than enzyme-based reactions, and the low yield of O-linked oligosaccharides that results from chemically-based cleavage methods, has hindered the use of high sensitivity MS measurements for oligosaccharides. The traditional alkaline β-elimination method generates high-salt conditions that are incompatible with MALDI-MS measurements. The desalting procedures, such as reacting the excess NaBH₄ with acetic acid and passing the sample through a large cation exchange column or the use of extensive dialysis, can result in substantial loss of oligosaccharides in the sample.

Accordingly, a new method of cleaving O-linked oligosaccharides from glycoproteins has been developed. As shown in FIG. 1, in accordance with the method of the present invention, oligosaccharide products form alditols upon cleavage from glycoproteins (represented in FIG. 1 by the GalNAc structure) and upon the reduction of the oligosaccharide products in the presence of borane-ammonia. The method of the present invention has been applied successfully in the analysis of oligosaccharides of bovine fetuin and submaxillary mucin, as well as the complex bile-salt-stimulated lipase isolated from human milk (see discussion below).

The steps in the β-elimination procedure of the present invention are different from the conventional alkaline β-elimination method in many aspects. First, aqueous sodium hydroxide has been replaced with aqueous ammonia. The ammonia maintains the alkaline pH (e.g., pH 8-12) which is needed for β-elimination of O-linked glycans while introducing the advantage of easy reagent removal through evaporation. Evaporation can be effected by any method known in the art including, for example, evaporation under a nitrogen stream. In the method of the present invention, the composition containing glycoproteins can be an aqueous composition and the water can be evaporated, for example, under a nitrogen stream, prior to addition of the solution comprising the borane-ammonia complex. The borane-ammonia complex can also be in an aqueous solution and the ammonia can be evaporated, for example, under a nitrogen stream, prior to reconstitution of the sample for separation to remove the protein by-products (e.g., protein or peptide components or fragments thereof) of the glycoprotein for analysis of the oligosaccharide products (e.g., by MS).

The second modification to the method of the present invention relative to the conventional alkaline B-elimination protocol is the elimination of sodium borohydride, an effective reducing agent but a major contributor to salt formation, and its replacement with borane-ammonia as a reducing agent. A reducing agent is advantageous because the reducing agent prevents degradation of the oligosaccharides released from glycoproteins that normally occurs due to “peeling reactions” (as previously observed with maltopentaose treated with an ammonia solution for 2 hours). The reducing agent chosen was borane-ammonia which serves as an effective reducing agent for aldehydes and ketones and possesses remarkable stability and handling convenience. The borane-ammonia complex (BH₃.NH₃) also offers a significantly greater rate of reduction than sodium borohydride and a higher efficiency of hydride delivery to sterically more hindered carbonyl groups in aldehydes and ketones. Additionally, the ability of ammonia to form a complex with borane may stabilize this reducing reagent permitting a significant reduction in the needed quantity for conversion leading to a further minimization of the subsequent cleanup steps.

Accordingly, in the method of the present invention a composition comprising a glycoprotein, wherein the glycoprotein comprises an O-linked oligosaccharide, is contacted with a solution comprising a borane-ammonia complex to form a mixture comprising the glycoprotein and the borane-ammonia complex.

The composition comprising a glycoprotein can contain one type of glycoprotein or can comprise more than one type of glycoprotein. The protein component of the glycoprotein can be a protein, a peptide, or a fragment of either a protein or peptide. The glycoprotein composition can be in an aqueous form or can be in any other form that is compatible with the β-elimination method of the present invention. Also, the glycoprotein composition can contain only the glycoprotein or can contain other components and the glycoprotein in the composition can be previously purified or unpurified.

The solution comprising the borane-ammonia complex can be in aqueous form or in any form compatible with the β-elimination method of the present invention. The solution comprising the borane-ammonia complex can comprise only the borane-ammonia complex or can comprise other components. The solution comprising the borane-ammonia complex can be added in any volume, but small volumes are preferable (e.g., about 5 μL to about 30 μL). As discussed above, a reduction in the amount of borane-ammonia needed for the reaction minimizes the subsequent clean up steps. Accordingly, although low concentrations are preferable (see Example 2 for exemplary concentrations), any concentrations compatible with the β-elimination method of the present invention can be used.

The mixture initially comprising the glycoprotein(s) and the borane30 ammonia complex is incubated for a period of time sufficient to cleave O-linked oligosaccharides from glycoproteins. Any period of time and any incubation temperature sufficient to cleave O-linked oligosaccharides from glycoproteins is contemplated in accordance with the invention. Exemplary of time periods for the incubation step are about 1 hour to about 24 hours. Exemplary temperature ranges for the incubation step are about 40° C. to about 50° C., about 35° C. to about 55° C., about 30° C. to about 60° C., and about 20° C. to about 60° C.

The incubation step can be carried out in any vessel, but a small vessel to hold small volumes, such as a microfuge tube, is preferred. The vessel (e.g., a microfuge tube) can be incubated in a water bath to achieve the desired temperature and the vessel can be held in a floating device. Preferably, the vessel (e.g., microfuge tube) is held in a position (e.g., upside down) so that the reaction mixture is as far away as possible, in the upwards direction, from the heat source so that incubation can be carried out without complete evaporation of the fluid in the reaction mixture.

Any method known in the art can be used to eliminate the excess borane-ammonia complex from the sample. For example, the absence of sodium borohydride from the β-elimination reaction permits the use of a small amount (e.g., about 10 to about 100 μL) of H⁺ cation exchanger to eliminate the excess borane-ammonia complex. In the method of the present invention, another highly hydrophobic resin (SP20SS) can also be utilized to retain the proteins or peptides, or fragments thereof, released during the β-elimination reaction which could potentially interfere with the subsequent analysis (e.g., MS measurements). Sample losses are significantly reduced by employing the minimum volumes of resins required, but any resin volumes can be used.

Any separation techniques known in the art can also be used such as capillary electrophoresis, capillary electrochromatography (e.g., polyethylene/polyacrylamide glycol matrices), anion exchange chromatography, HPLC, microcolumn size exclusion, other miniturized separation techniques, and the like. These separation techniques can be used for separating cleaved oligosaccharide products from other cleaved oligosaccharide products or for separating cleaved oligosaccharide products from protein by-products of the β-elimination reaction.

The oligosaccharide products of the β-elimination reaction can be analyzed by any technique known in the art. Exemplary techniques include mass spectrometry techniques such as MALDI/MS, MALDI/TOF MS, and electrospray ionization MS using an ion trap, and high-field NMR, and the like. MALDI matrices that can be used include arabinosazones and other osazones, cationic substances, spermine as a co-matrix with DHB, and the like. Any other matrix known in the art can also be used. The sample preparation preferably provides the ability to analyze both neutral and acidic oligosaccharides from a single spot by simply switching the instrument between the positive- and negative-ion modes.

Reduction of Dextrans by BH₃.NH₃

The method developed in accordance with the present invention was first tested using a dextran ladder sample. It was expected that conversion of the reducing oligosaccharide series in dextrans to alditols would increase their molecular weights by two mass units. For dextrans, treated in accordance with the method of the present invention, the conversion of reducing oligosaccharides to alditols was determined to be quantitative irrespective of the oligosaccharide size. Additionally, the reaction was substantially complete, as no signals originating from the residual reducing oligosaccharides were observed (i.e., the oligosaccharides had been converted to alditols). Furthermore, no “peeling reactions” were evident in the spectrum. Thus, the method of the present invention was applied to the cleavage of O-glycans from glycoproteins.

O-Glycan Cleavage from Glycoproteins

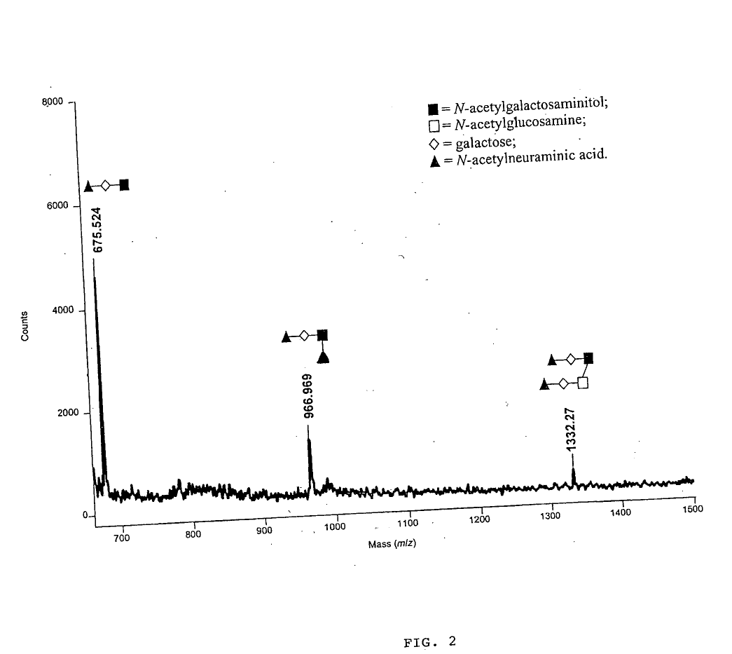

The oligosaccharides in fetuin isolated from calf serum have been previously characterized. Therefore, fetuin was initially used to test the method of the present invention. As shown in FIG. 2, the three oligosaccharides known to exist in fetuin were observed in the MALDI mass spectrum of fetuin treated in accordance with the method of the invention. Since all of the oligosaccharides in fetuin are known to be sialylated structures, the negative-ion mode was employed in MALDI/MS. Indeed, the observed 675.52, 966.97, and 1332.27 m/z values represent the molecular ions of [M-H]⁻ corresponding to the in-, tetra-, and pentaoligosaccharides in fetuin, respectively.

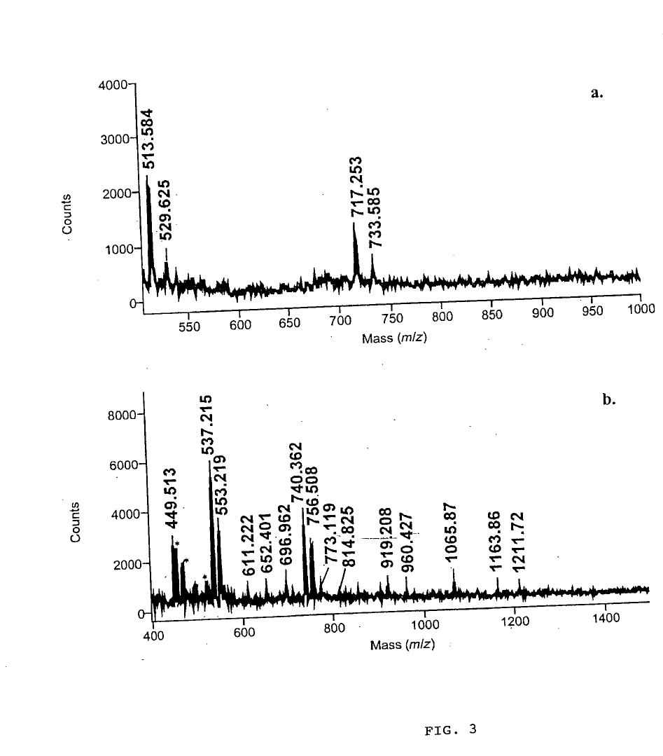

The oligosaccharides cleaved from bovine submaxillary mucin have been studied using various analytical techniques, including the traditional β-elimination procedure employing ammonium hydroxide, sodium borohydride and amounts of material ranging from milligrams to grams. In these studies, a great diversity of oligosaccharides in mucin was generally observed. Both neutral and acidic oligosaccharides were observed, ranging from monosaccharide to octasaccharide structures and occurring at different concentrations. Using the method in accordance with the present invention, abundant signals were obtained from just 5 μg of mucin, and the major oligosaccharide components were observed (see FIG. 3). The strong signals in the negative-ion mass spectrum (see FIG. 3a) correspond to the major acidic oligosaccharides, namely, NeuAca(2→6)GalNAc-ol, NeuGcα(2→6)GalNAc, GlcNAcβ(1→3)[NeuAcα(2→6)]GalNac-ol, and GlcNAcβ(1→3) [NeuGcα(2→6)]GalNAc-ol. Other acidic components of mucin were not observed due to their low concentrations. Sodium adduct ions of the oligosaccharide components of mucin were also observed as major signals when the same sample spot was analyzed in the positive-ion mode (see FIG. 3b). Additionally, the neutral oligosaccharide components of mucin were observed. Both the acidic and neutral oligosaccharides observed in mucin using the method in accordance with the present invention are shown in Table 1.

TABLE 1
Saccharide composition of the O-glycans
released from bovine mucin analyzed by MS.

| [M − H]⁻ | [M − H]⁻ | [M + NA]⁺ | [M + NA]⁺ | |
|---|---|---|---|---|
| Observed | Calculated | Observed | Calculated | Saccharide Composition |
| 513.584 | 513.45 | 537.215 | 537.45 | NeuAc GalNAc-ol |
| 529.625 | 529.45 | 553.219 | 553.45 | NeuGc GalNAc-ol |
| 717.253 | 716.65 | 740.362 | 740.65 | GlcNAc (NeuAc) GalNAc-ol |
| 733.585 | 732.65 | 756.508 | 756.65 | GlcNAc (NeuGe) GalNAc-ol |
| | | 449.513 | 449.39 | GlcNAc GalNAc-ol |
| | | 611.222 | 611.53 | Gal GlcNAc GalNAc-ol |
| | | 652.401 | 652.59 | GlcNAc₂ GalNAc-ol |
| | | 756.508 | 757.68 | Fuc Gal GlcNAc GalNAc-ol |
| | | 773.119 | 773.67 | Gal₂ G1cNAc GalNAc-ol |
| | | 814.825 | 814.73 | Gal GlcNAc₂ GalNAc-ol |
| | | 919.208 | 919.82 | Fuc Gal₂ GlcNAc GalNAc-ol |
| | | 960.427 | 960.87 | Fuc Gal GlcNAc₂ GalNAc-ol |
| | | 1065.87 | 1065.96 | Fuc₂ Gal₂ GlcNAc GalNAc-ol |
| | | 1163.86 | 1164.07 | Fuc Gal GlcNAc₃ GalNAc-ol |
| | | 1211.72 | 1212.10 | Fuc₃ Gal₂ GlcNAc GalNAc-ol |

Finally, the effectiveness of the method of the present invention was demonstrated by cleaving O-linked oligosaccharides from the bile-salt-stimulated lipase (BSSL) isolated from human milk. This 100 kDa glycoprotein has a threonine- and serine-rich region at the C-terminus, suggesting a potentially high number of O-liked oligosaccharides and, consequently, a complex O-glycan pool. BSSL was previously found to express the Lewis antigens (a, b, and x) when monoclonal antibodies were used. The oligosaccharide components of BSSL are important because they likely have biological activities (e.g., antimicrobial) beneficial to the infant.

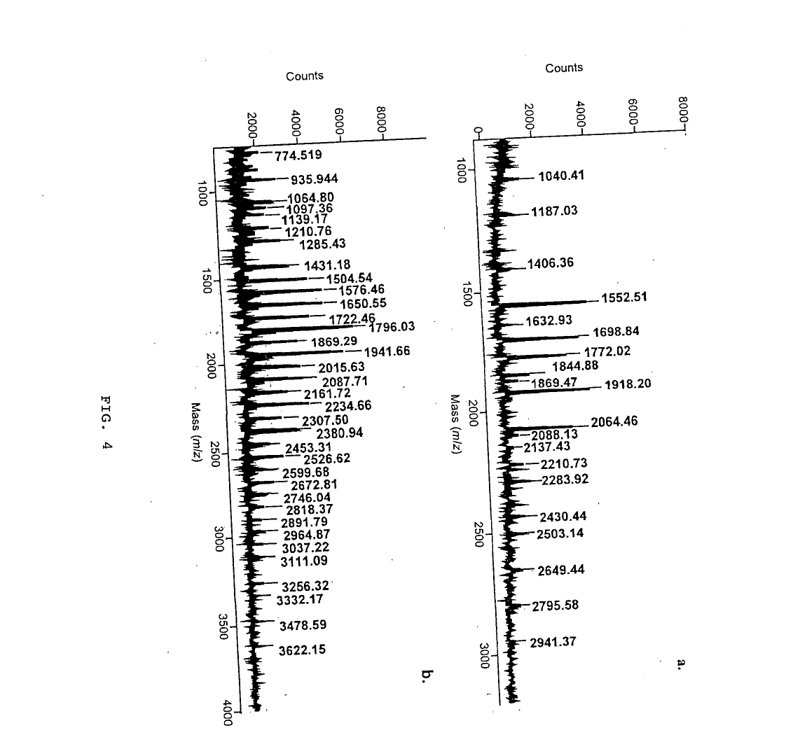

Using 1 μg of BSSL treated according to the method of the present invention to effect β-elimination, a very high heterogeneity of oligosaccharides was observed during a MALDI-MS analysis. As shown in FIG. 4a, there are more than a dozen sialylated oligosaccharides detected in BSSL using MALDI/MS in the negative-ion mode. Moreover, the m/z difference of 146 between many signals (e.g., m/z 1040.41 vs. 1187.03, 1406.36 vs. 1552.51 and 1698.84, 1772.02 vs. 1918.2 and 2064.46) suggested that fucosylation is a common process in the course of glycan biosynthesis. The positive-ion mass spectrum (see FIG. 4b) exhibited even more heterogeneity, as both neutral and acidic oligosaccharides were detected and extensive fucosylation of the oligosaccharides was observed.

Thus, a novel β-elimination procedure has been developed, permitting an effective and facile chemical cleavage of O-linked oligosaccharides from low- to sub-microgram amounts of glycoproteins. Borane-ammonia complex in aqueous ammonia was used as the release medium, replacing the sodium borohydride/sodium hydroxide solution used in the conventional alkaline β-elimination reduction. Both the residual ammonia and borane-ammonia complex can be easily removed through evaporation, reducing significantly the sample handling problems after the cleavage reaction. Thus, the procedure results in minimum sample purification and sample loss, and, consequently, an enhancement of the capacity for structural analysis of oligosaccharides by MS. The method of the present invention has been applied successfully to several glycoproteins, including calf serum fetuin and bovine submaxillary mucin, as well as the much more complex bile-salt-stimulated lipase isolated from human milk.

In accordance with the method of the present invention the released glycans are converted to alditols and cannot be easily tagged with a chromophore or fluorophore for sequencing by MALDI/MS, for example. However, because the present method results in an enhancement of the capacity of structural analysis by MS, the present method is advantageous in applications where derivatization is not necessary.

### EXAMPLE 1

### Materials for Preparation of Oligosaccharides

Bovine submaxillary mucin type 1S, fetal calf serum fetuin, spermine tetrahydrochloride, and DOWEX 50W X8-400 cation exchanger (H⁺ form) were purchased from Sigma Chemical Company (St. Louis, Mo.). Sepabeds™ SP20SS resin was obtained from Supelco, Inc. (Bellefonte, Pa.). Both the cation exchanger and SP20SS resin were pre-conditioned according to the manufacturers' manuals. Dextran (MW 1500) was from Fluka Biochemika (Buchs, Switzerland). 2,5-Dihydroxybenzoic acid (DHB) and borane-ammonia complex were purchased from Aldrich Chemical Company (Milwaukee, Wis.). High-purity water was a product of Criterion Science (Riverdale, N.J.). Ammonium hydroxide (28-30%) was obtained from J. T. Baker, Inc. (Phillipsburg, N.J.). The bile salt-stimulated lipase (BSSL) sample from human milk was provided by Professor Peter Pahlsson, Department of Clinical Chemistry, University Hospital, Linkoping, Sweden.

### EXAMPLE 2

### β-Elimination of Dextran

A dextran preparation was dissolved in water at 1 mg/mL. A 1 μL aliquot was added to a 600 μL microtube and dried under a stream of nitrogen. A 10 μL aliquot of borane-ammonia complex solution (prepared at 5 mg/mL in a 28% aqueous ammonia solution) was then added, and the reaction mixture was incubated at 45° C. for 1 h. The mixture was subsequently dried under a nitrogen stream to remove the ammonia, and the residue was reconstituted in 20 μL of high-purity water. The solution was loaded onto a microcolumn made from a micropipette tip packed with a 20 μL volume of the cation-exchange resin DOWEX 50W X8-400 (H⁺ form). A 200-μL volume of aqueous eluent was collected and lyophilized. The residual boric acid was removed through several additions of 200 μL of methanol with evaporation after each addition. The final product was dissolved in 4 μL of high-purity water and then subjected to MS analysis.

### EXAMPLE 3

### O-Linked Oligosaccharide Cleavage Protocol

Typical glycoprotein samples, such as calf serum fetuin, bovine submaxillary mucin, and human milk bile salt-stimulated lipase, were prepared as aqueous solutions at 10 mg/mL concentrations. Small aliquots (e.g., 1-5 μL) were transferred to a microtube and dried under nitrogen. A 10 μL aliquot of the borane-ammonia complex solution (prepared as described in Example 2) was then added. The mixture was subsequently incubated at 45° C. for about 18-24 h. The reaction mixtures were then purified as described for the dextran samples in Example 2, except that the microcolumns were prepared with a 20 μL volume of SP20SS resin (bottom of column) and a 40 μL volume of the cation-exchanger (DOWEX 50W X8-400 (H⁺ form); top of column). The eluent was subjected to MS analysis.

### EXAMPLE 4

### MALDI/TOF Mass Spectrometric Measurements

All experiments were carried out using a Voyager-DE™ RP BioSpectrometry Workstation MALDI/time-of-flight (TOF) mass spectrometer (PerSeptive Biosystems, Framingham, Mass.) with a pulsed nitrogen laser (337 nm). The instrument has a mass precision of 0.1% with external calibration. The sample preparation was a modified version of a previously published procedure (Rapid Commun. Mass Spectrom. 14:1233-1237 (2000)). Briefly, a DHB/spermine matrix solution was prepared by mixing a 500 μL aliquot of 20 mg/mL DHB in acetonitrile and a 500 μL aliquot of 25 mM aqueous spermine solution. A sample spot was prepared by mixing a sample solution and 1 μL DHB/spermine matrix solution on the stainless steel sampling plate, followed by drying in vacuuo. The sample was measured in both the positive- and negative-ion mode. In the positive-ion mode experiments, the instrument was calibrated externally with a dextran ladder, while in the negative-ion mode, the oligosaccharides released from fetuin were used as calibrants. MALDI/TOF MS analysis of oligosaccharides from calf serum fetuin, bovine submaxillary mucin, and human milk bile salt-stimulated lipase is shown in FIGS. 2-4.

What is claimed is:

1. A method of cleaving an O-linked oligosaccharide from a glycoprotein, said method comprising the steps of
contacting a composition comprising a glycoprotein, wherein the glycoprotein comprises O-linked oligosaccharides, with a solution comprising a borane-ammonia complex to form a mixture comprising the glycoprotein and the borane-ammonia complex;
incubating the mixture for a period of time sufficient to cleave the linked oligosaccharides from the glycoprotein; and
forming a mixture comprising oligosaccharide alditol products and deglycosylated protein by-products.

2. The method of claim 1 further comprising the step of separating at least one cleaved oligosaccharide product from the other oligosaccharide products.

3. The method of claim 1 further comprising the step of separating at least one cleaved oligosaccharide product from the protein by-products.

4. The method of claim 2 or 3 further comprising the step of analyzing the structure of the oligosaccharide product.

5. The method of claim 4 wherein the structure of the cleaved oligosaccharide is analyzed by mass spectrometry.

6. The method of claim 5 wherein the mass spectrometry method is selected from the group consisting of matrix-assisted laser desorption ionization mass spectrometry and matrix-assisted laser desorption ionization/time-of-flight mass spectrometry.

7. The method of claim 2 or 3 wherein the separation is achieved using a cation exchange resin.

8. The method of claim 2 or 3 wherein the separation is achieved using a hydrophobic resin.

9. The method of claim 2 or 3 wherein the separation is achieved using a cation exchange resin and a hydrophobic resin.

10. The method of claim 1 wherein the incubation step is performed a temperature of about 40° C. to about 50° C.

11. The method of claim 1 wherein the incubation step is performed a temperature of about 35° C. to about 55° C.

12. The method of claim 1 wherein the incubation step is performed a temperature of about 30° C. to about 60° C.

13. The method of claim 1 wherein the incubation step is performed a temperature of about 20° C. to about 60° C.
